(19) United States
(12) Patent Application Publication
(10) Pub. No.: US 2014/0235472 A1
(43) Pub. Date: Aug. 21, 2014

(54) METHODS AND COMPOSITIONS FOR THE DETECTION OF BALANCED RECIPROCAL TRANSLOCATIONS/REARRANGEMENTS

(71) Applicant: UNIVERSITY OF UTAH RESEARCH FOUNDATION, Salt Lake City, UT (US)

(72) Inventors: Shashirekha Shetty, North Olmsted, OH (US); Rong Mao, Salt Lake City, UT (US)

(73) Assignee: UNIVERSITY OF UTAH RESEARCH FOUNDATION, Salt Lake City, UT (US)

(21) Appl. No.: 14/118,843

(22) PCT Filed: May 18, 2012
(86) PCT No.: PCT/US2012/038674

§ 371 Date: May 5, 2014

Related U.S. Application Data
(60) Provisional application No. 61/487,791, filed on May 19, 2011.

(51) Int. Cl. C12Q 1/68

(52) U.S. Cl. CPC C12Q 1/6837
USPC 506/9; 506/16

(57) ABSTRACT

Disclosed are methods and compositions related to the detection of balanced reciprocal translocations/rearrangements and determination of the location of said balanced reciprocal translocations/rearrangements. Also disclosed are methods and compositions relating to the diagnosis of a disease or condition associated with uncontrolled cellular proliferation by the detection of balanced reciprocal translocation/rearrangement. Additionally, also disclosed are kits for the detection of balanced reciprocal translocations/rearrangements.

### I. CROSS REFERENCE TO RELATED APPLICATIONS

This application claims the benefit of U.S. Provisional Patent Application Ser. No. 61/487,791, filed on May 19, 2011, which application is hereby incorporated by this reference in its entirety.

### II. BACKGROUND

Balanced reciprocal translocations/rearrangements involve an aberrant fusion of genes with no gain or loss of genetic material. Such translocations have been linked to several forms of cancer with approximately 300 genes involved in fusions leading to both benign and malignant neoplastic disorders. The first specific balanced reciprocal translocation/rearrangement identified in human cancers was t(9;22)(q34;q11), resulting in the Philadelphia chromosome (derivative chromosome 22). The molecular characterization of t(9;22) in chronic myelogeneous leukemia (CML) revealed a fusion between the ABL1 gene on chromosome 9 and the BCR gene on chromosome 22 with no gain or loss of genetic or DNA (deoxynucleotide) material, dramatically increased our understanding of the pathogenetic significance of translocations and gene fusions in the origin of neoplasia. This understanding lead to the use of cytogenetics as a valuable tool to map genes associated with oncogenesis. As a consequence, the information on chromosome aberrations in cancers has steadily increased over the past decades. Such cytogenetic characterization has been of the greatest importance in detecting genes associated with tumorigenesis and has, to date, led to the identification of approximately 300 genes involved in fusions in both benign and malignant neoplastic disorders. These genes represent a substantial proportion of all mutated genes that have been implicated in oncogenesis.

Comparative genomic hybridization (CGH) is perhaps the most significant technical development in the molecular cytogenetics period, and has contributed considerably to our further understanding of the cancer genome. Over recent years, the chromosome study has been largely superseded by array CGH (aCGH) in which changes in copy number can be mapped to the DNA sequence at a high resolution. However; one of the major problems encountered with aCGH is that it cannot detect balanced translocations/rearrangements as is the case with translocations or fusion genes; with no gain or loss at the DNA sequence level. Till date all the microarray platforms that available for cancer cytogenetics markers can only target copy number changes i.e., losses or gains of genetics material and will not detect presence of fusion gene sequences as these are balanced. So far, over 300 fusion genes have been identified in cancers and the first designer drug and most effective therapy for CML i.e., Imagine (Gleeman) was based on our knowledge of fusion of the BCR and ABL1 genes. Given recent improvements in quality and expense of custom designed microarrays, our goal to implement a sensitive and cost-effective assay to detect balanced reciprocal translocations/rearrangements is achievable.

### III. SUMMARY

Disclosed are methods and compositions related to the detection of balanced reciprocal translocations/rearrangements and determination of the location of said balanced reciprocal translocations/rearrangements. Also disclosed are methods and compositions relating to the diagnosis of a disease or condition associated with uncontrolled cellular proliferation by the detection of balanced reciprocal translocation/rearrangement. Additionally, also disclosed are kits for the detection of balanced reciprocal translocations/rearrangements.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows tiled oligonucleotide probes (60 mers in length) tiled in a 4-7 bp spacing for the 50 targeted genes and 500 bp upstream and downstream regions.

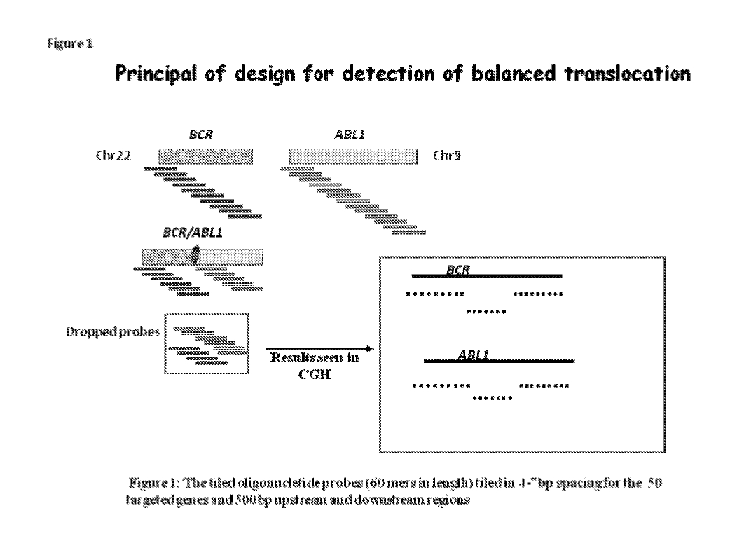

FIG. 2 shows at(9;22) BCR/ABL1 translocation detected in sample C by the BCRONCO array. FIG. 2A shows the “U or crescent” shape pattern involving 19 probes in the size of 90 bp with Log 2 Ratio of −0.86527 in the intron 1 of ABIL gene. FIG. 2B shows the “U or crescent” shape pattern of 28 probes drop off in the size of 138 bp in the intron 15 of the BCR gene. FIG. 2C shows a diagram showing the translocation of BCR intron 15 to ABL1 intron 1 detected in sample C.

FIG. 3 shows a t(6;9) DEK/NUP214 translocation detected in sample 1 by the BCRONCO array. FIG. 3A shows the “U or crescent” shape pattern involving 10 probes in the size of 44 bp with Log 2 Ratio of −0.73717 in intron 21 of NUP214 gene. FIG. 3B shows the “U or crescent” shaped pattern of 17 probes drop off in the size of 78 bp in intron 9 of the DEK gene. FIG. 3C shows a diagram showing the translocation of NUP214 intron 21 in DEK intron 9 detected in sample 1.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings.

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF DETECTING CHROMOSOMAL REARRANGEMENTS

Large scale genomic aberrations, including balanced rearrangements (translocations and inversions) and genomic imbalances (deletions, duplications, and amplifications) are common in cancer and play central roles in oncogenesis. Genomic deletions typically have been associated with loss of tumor suppressor gene function, amplifications with over-expression of proto-ontogenesis, and translocations with the creation of novel monogenic gene fusions or deregulated ontogeny expression. Historically, balanced translocations/rearrangements and gene fusions have been seen predominantly in hematologic and mesenchymal tumors, including sarcomas, leukemias, and lymphomas, and less commonly in epithelial tumors such as carcinomas. More recently, oncogenic gene fusions have been identified in carcinomas of the prostate, thyroid, and lung, suggesting that they are more common in carcinomas than previously appreciated, possibly because of the cytogenetic complexity of these tumors or for other technical reasons.

Analysis of large scale aberrations has transformed our understanding of oncogenesis through the identification of novel proto-oncogenes, tumor suppressor genes, and oncogenic gene fusions. Advances in microarray-based comparative genomic hybridization (array-CGH/aCGH) technology have led to accumulation of data on genomic imbalances in cancer, which can be mapped at ever increasing resolution. A minority of translocations have small deletions at one or both breakpoints that can be identified by arrayCGH. However, such imbalances are not usually detectable and, even when present, they do not indicate the identity of the other partner gene.

Structural variation is now recognized as an important source of genetic variation across human populations. Copy number variants (CNV) like chromosomal deletions or segmental duplications represent the most common structural variants reported to date and have been associated with human disease or predisposition to disease. Methods for identifying balanced chromosomal rearrangements have lagged behind those for detecting CNV, but chromosomal inversions and more complex rearrangements are increasingly recognized as important sources of structural and functional variation and also have been associated with human disease.

Currently, methods designed to detect balanced rearrangements are limited when compared with methods available to detect genomic imbalances. Traditional cytogenetic analysis and multicolor (or spectral) karyotyping are powerful genome-wide techniques for identifying large-scale genomic abnormalities, but these methods are laborious, require growth of cells in culture, and have limited resolution (5-10 million bp). Fluorescence in situ hybridization (FISH) can be used to analyze small numbers of genomic loci at higher resolution (typically 100-1000 kb), but FISH is not readily scalable and requires prior knowledge of the fusion partners. In array painting, DNA is amplified from flow-sorted abnormal chromosomes and hybridized to CGH arrays, enabling translocation breakpoints to be mapped to high resolution

Array based comparative genomic hybridization (CGH) has revolutionized the study of chromosomal imbalances but generally is incapable of detecting balanced genomic rearrangements like reciprocal translocations, which play central roles in the pathogenesis and diagnosis of lymphomas, leukemias and other tumors. Accordingly, disclosed herien are high density tiling genomic microarray chip based tests for the detection of balanced reciprocal translocations/rearrangements in diseases and disorders with uncontrolled cellular proliferation such as cancers and neoplastic disorders. Thus, in one embodiment, disclosed herein are methods of detecting the presence of a chromosomal rearrangement in a test sample comprising (a) isolating a first genomic DNA from cells of a test sample and a second genomic DNA from cells of a reference sample; (b) amplifying the DNA from the test sample and reference sample; (c) hybridizing the amplified test and reference DNA products to a tiling density DNA microarray comprising genomic DNA sequences; and (d) comparing the pattern and extent of hybridization of the test amplified DNA product with the reference amplified DNA product to the DNA microarray.

As used herein, “chromosomal rearrangement” or “chromosomal abnormality” refers generally to the aberrant joining of segments of chromosomal material in a manner not found in a wild-type or normal cell. Examples of chromosomal rearrangements include deletions, amplifications, inversions, or translocations. Chromosomal rearrangements can arise after spontaneous breaks occur in a chromosome. If the break or breaks result in the loss of a piece of chromosome, a deletion has occurred. An inversion results when a segment of chromosome breaks off, is reversed (inverted), and is reinserted into its original location. When a piece of one chromosome is exchanged with a piece from another chromosome a translocation has occurred. Amplification results in multiple copies of particular regions of a chromosome. Chromosomal rearrangements may also encompass combinations of the above.

As used herein “translocation” or “”chromosomal translocation” refers generally to chromosomal rearrangement involving an exchange of chromosomal material between the same or different chromosomes in equal or unequal amounts. Frequently, the exchange occurs between nonhomologous chromosomes. A “balanced” translocation/rearrangement refers generally to an exchange of chromosomal material in which there is no net loss or gain of genetic material. Therefore, in one aspect, the disclosed methods can be used to detect the presence and location of balanced translocations/rearrangements occurring in a test sample. Thus, for example, disclosed herein are methods of detecting the presence of a balanced reciprocal translocation/rearrangement in a test sample comprising (a) isolating a first genomic DNA from cells of a test sample and a second genomic DNA from cells of a reference sample; (b) amplifying the DNA from the test sample and reference sample; (c) hybridizing the amplified test and reference DNA products to a tiling density DNA microarray comprising genomic DNA sequences; and (d) comparing the pattern and extent of hybridization of the test amplified DNA product with the reference amplified DNA product to the DNA microarray.

In general, the methods of the present disclosure can be used to detect and map chromosomal abnormalities or rearrangements, such as, for example, balanced reciprocal translocations/rearrangements. In one embodiment, a method of the present methods utilize a first population of genomic nucleic acids obtained from a test sample, such as a patient sample, and a second population of genomic nucleic acids obtained from a reference sample. The reference sample may be any cells, tissues or fluid as provided herein, obtained from an individual, or any cell culture or tissue culture, that does not contain any genetic abnormality, i.e., that has a normal genetic complement of all chromosomes.

Although not necessary, the present methods can employ the use of primers specific to a particular genomic locus to perform linear amplification of sequences encompassed by the genomic locus and extending into the sequence of a translocation partner to generate a probe molecule that includes both members of a translocation pair. At the same time, a reference probe can also be generated using linear amplification of genomic DNA from a reference cell in the manner described for the test sample. Regardless of the use or not of primers and amplification, the test and reference probes are differentially labeled, for example, with a fluorescent label such as Cy3 and Cy5, although many suitable fluorescent label pairs are known in the art. The differentially labeled probes are then hybridized to microarrays comprising genomic DNA. Generally, the sequences of the genomic DNA of the microarray are derived from a reference source such as database sequences for a particular organism, e.g., the complete database of the human, mouse, or rat genome. The pattern and extent of hybridization of the test sample probe as compared to the hybridization of a similar probe derived from a reference sample allows the identification of the translocation partner of the known genomic locus. The use of high density microarrays, such as tiling density microarrays, allows high resolution mapping of the breakpoints of the translocation.

Accordingly, if a translocation is present at a genomic locus of interest, hybridization of the test probe to a microarray comprising genomic DNA sequences from a reference cell will result in signal associated with elements corresponding to the known genomic locus as well as signals associated with elements of the microarray associated with another genomic locus. The signal associated with the other genomic locus identifies that locus as being a partner of the known genomic locus regardless of the rearrangement type i.e., translocation or inversion or any balanced rearrangement. In contrast, hybridization of the microarray with the reference probe will result in hybridization exclusively associated with microarray elements corresponding with the known locus, with no hybridization signal associated with another genomic locus as observed with the test probe.

In one embodiment, this array detects variable breakpoints associated with the reciprocal translocations commonly described in neoplasia especially in hematological malignancies and solid tumors. It is understood and herein contemplated that the there are many variations on the disclosed methods that can be used to detect balanced translocations/rearrangements. One example of such a method is the use of a high density tiling array.

High density tiling microarrays can ascertain the breakpoints of the translocation by determining where hybridization commences and ends in a series of microarray elements embodying overlapping contiguous segments of genomic DNA. Thus, the cessation of hybridization at a specific point along a series of elements corresponding to the known genomic locus using the test probe, with hybridization continuing along the series using the reference probe, identifies the point at which hybridization stops as being the translocation breakpoint for the known genomic locus. Similarly, the point at which hybridization by the test probe commences in a series of elements corresponding to a locus distinct from the known genomic locus, and which is negative for hybridization by the reference probe, indicates that the first element at which hybridization occurs is the breakpoint for the translocation partner of the known genomic locus. This patterned loss and recovery of hybridization which occurs at the breakpoint of translocation is referred to as “drop off” and has a distinct “U or crescent” shape. Accordingly, detection of a “U or crescent” shape pattern is thus indicative of the detection of a balanced reciprocal translocation/rearrangement. In a typical experiment, genomic DNA from a lymphoma and from a normal control undergoes whole genome amplification prior to being applied to the chip array although it is also contemplated herein that the amplification can be linear amplification. When linear amplification and fluorescent labeling, and is then combined and hybridized to a custom oligonucleotide array. The breakpoints are identified on arrays by virtue of their characteristic U or crescent shape. Therefore, disclosed herein are methods of detecting the presence and/or location of a chromosomal rearrangement such as a balanced reciprocal translocation wherein a patterned loss and recovery of hybridization of the test sample DNA product over the reference sample DNA product to a DNA microarray element indicates the presence of chromosomal rearrangement.

As used herein, a high density tiling microarray chip such as, for example, a balanced rearrangement oncology chip (BCRONC/RS) can detect balanced translocations/rearrangements not detected by current single nucleotide polymorphism (SNPs) arrays and array comparative genomic hybridization (aCGH) platforms. In addition, copy number changes in the genome for targeted regions is also be detected. This assay is able to replace current multi-steps, expensive FISH and microarray tests for oncology especially in hematological malignancies and solid tumors.

1. Biological Samples

In one aspect, the methods of the present invention can be used to detect a chromosomal abnormality in a test sample. Generally, the test sample is obtained from a patient. The test sample can contain cells, tissues, or fluid obtained from a patient suspected of having a pathology or a condition associated with a chromosomal or genetic abnormality. For the purposes of diagnosis or prognosis, the pathology or condition is generally associated with genetic defects, e.g., with genomic nucleic acid base substitutions, amplifications, deletions and/or translocations. The test sample may be suspected of containing cancerous cells or nuclei from such cells. For example, the test sample can be a cell from a tumor such as a lymphoma or a cell associated with leukemia.

Samples may include, but are not limited to, amniotic fluid, biopsies, blood, blood cells, bone marrow, cerebrospinal fluid, fecal samples, fine needle biopsy samples, peritoneal fluid, plasma, pleural fluid, saliva, semen, serum, sputum, tears, tissue or tissue homogenates, tissue culture media, urine, and the like. Samples may also be processed, such as sectioning of tissues, fractionation, purification, or cellular organelle separation.

Methods of isolating cell, tissue, or fluid samples are well known to those of skill in the art and include, but are not limited to, aspirations, lavage, tissue sections, drawing of blood or other fluids, surgical or needle biopsies, and the like. Samples derived from a patient may include frozen sections or paraffin sections taken for histological purposes. The sample can also be derived from supernatants (of cell cultures), lysates of cells, cells from tissue culture in which it maybe desirable to detect levels of mosaicisms, including chromosomal abnormalities, and copy numbers.

In one embodiment, a sample suspected of containing cancerous cells is obtained from a human patient. Samples can be derived from patients using well-known techniques such as venipuncture, lumbar puncture, fluid sample such as saliva or urine, tissue or needle biopsy, buccal swabs and the like. In a patient suspected of having a tumor containing cancerous cells, a sample may include a biopsy or surgical specimen of the tumor, including for example, a tumor biopsy, a fine needle aspirate, or a section from a resected tumor. A lavage specimen may be prepared from any region of interest with a saline wash, for example, cervix, bronchi, bladder, etc. A patient sample may also include exhaled air samples as taken with a breathalyzer or from a cough or sneeze. A biological sample may also be obtained from a cell or blood bank where tissue and/or blood are stored, or from an in vitro source, such as a culture of cells. Techniques for establishing a culture of cells for use as a sample source are well known to those of skill in the art.

2. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GeneChip® (Affymetrix, Inc which refers to its high density, oligonucleotide-based DNA arrays), and gene array.

DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the disclosed microarrays can be used to monitor gene expression, disease diagnosis, gene discovery, drug discovery (pharmacogenomics), and toxicological research or toxicogenomics.

Typically, a “nucleic acid array” or “nucleic acid microarray” is a plurality of nucleic acid elements, each comprising one or more target nucleic acid molecules immobilized on a solid surface to which probe nucleic acids are hybridized. Nucleic acids molecules that can be immobilized on such solid support include, without limitation, oligonucleotides, cDNAs, and genomic DNA. In the context of the present invention, microarrays containing sequences corresponding to different segments of genomic nucleic acids are used. The genomic elements of microarrays can represent the entire genome of an organism or else represent defined regions of a genome, e.g., particular chromosomes or contiguous segments thereof.

There are two variants of the DNA microarray technology, in terms of the property of arrayed DNA sequence with known identity. Type I microarrays comprise a probe cDNA (5005,000 bases long) that is immobilized to a solid surface such as glass using robot spotting and exposed to a set of targets either separately or in a mixture. This methods traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

To prepare beads coated with immobilized probes, beads are immersed in a solution containing the desired probe sequence and then immobilized on the beads by covalent or noncovalent means. Alternatively, when the probes are immobilized on rods, a given probe can be spotted at defined regions of the rod. Typical dispensers include a micropipette delivering solution to the substrate with a robotic system to control the position of the micropipette with respect to the substrate. There can be a multiplicity of dispensers so that reagents can be delivered to the reaction regions simultaneously. In one embodiment, a microarray is formed by using ink jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate (Baldeschweiler et al. PCT publication WO95/251116).

Samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. For example, methods of purification of nucleic acids are described in Laboratory Techniques in Biochemistry and Molecular Biology: Hybridization With Nucleic Acid Probes. Part I. Theory and Nucleic Acid Preparation, P. Tijssen, ed. Elsevier (1993). In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one preferred embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art (Wahl, G. M. and S. L. Berger (1987) Methods Enzymol. 152:399-407; Kimmel, A. R. (1987) Methods Enzymol. 152:507-511; Ausubel, F. M. et al. (1997) Short Protocols in Molecular Biology, John Wiley & Sons, New York, N.Y.; and Sambrook, J. et al. (1989) Molecular Cloning, A Laboratory Manual, Cold Spring Harbor Press, Plainview, N.Y.).

Methods for detecting complex formation are well known to those skilled in the art. In a preferred embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Type II microarrays comprise an array of oligonucleotides (2080-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GeneChip® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

Genome tiling microarrays comprise overlapping oligonucleotides designed to provide complete or nearly complete representation of an entire genomic region of interest.

Comparative genomic hybridization (CGH) refers generally to molecular-cytogenetic methods for the analysis of copy number changes (gains/losses) in the DNA content of a given subject's DNA and often in tumor cells. In the context of cancer, the method is based on the hybridization of labeled tumor DNA (frequently with a fluorescent label) and normal DNA (frequently with a second, different fluorescent label) to normal human metaphase preparations. Using epifluorescence microscopy and quantitative image analysis, regional differences in the fluorescence ratio of gains/losses vs. control DNA can be detected and used for identifying abnormal regions in the genome. CGH will generally detect only unbalanced chromosomes changes. Structural chromosome aberrations such as balanced reciprocal translocations or other balanced rearrangements that include inversions or insertions can not be detected, as they do not change the copy number. See, e.g., Kallioniemi et al., Science 258: 818-821 (1992).

In a variation of CGH, termed “Chromosomal Microarray Analysis (CMA)” or “ArrayCGH”, DNA from subject tissue and from normal control tissue (a reference) is differentially labeled (e.g., with different fluorescent labels). After mixing subject and reference DNA along with unlabeled human cot 1 DNA to suppress repetitive DNA sequences, the mixture is hybridized to a slide containing a plurality of defined DNA probes, generally from a normal reference cell. See, e.g., U.S. Pat. Nos. 5,830,645; 6,562,565. When oligonucleotides are used as elements on microarrays, a resolution typically of 20-80 base pairs can be obtained, as compared to the use of BAC arrays which allow a resolution of 100 kb. The (fluorescence) color ratio along elements of the array is used to evaluate regions of DNA gain or loss in the subject sample.

“Amplification” or an “amplification reaction” refers to any chemical reaction, including an enzymatic reaction, which results in increased copies of a template nucleic acid sequence. Amplification reactions include polymerase chain reaction (PCR) and ligase chain reaction (LCR) {see U.S. Pat. Nos. 4,683,195 and 4,683,202; PCR Protocols: A Guide to Methods and Applications (Innis et al, eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al, J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al, Genet. Anal. 15(2):35 (1999)) and branched DNA signal amplification (bDNA) {see, e.g., Iqbal et al, Mol Cell Probes 13(4):315 (1999)). As used herein, test and reference sample material can be subjected to whole genome amplification to increase the starting material for reaction. It is further contemplated that other amplification methods, such as, for example, linear amplification, are compatible with and can be used in the disclosed methods.

Linear amplification refers to an amplification reaction which does not result in the exponential amplification of DNA. Examples of linear amplification of DNA include the amplification of DNA by PCR methods when only a single primer is used, as described herein. See, also, Liu, C. L., S. L. Schreiber, et al., BMC Genomics, 4: Art. No. 19, May 9, 2003. Other examples include isothermic amplification reactions such as strand displacement amplification (SDA) (Walker, et al. Nucleic Acids Res. 20(7): 1691 (1992); Walker PCR Methods Appl 3(1): 1 (1993), among others.

The reagents used in an amplification reaction can include, e.g., oligonucleotide primers; borate, phosphate, carbonate, barbital, Tris, etc. based buffers {see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

Any known microarray and/or method of making and using microarrays can be used in the practice of the present invention, such as those disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8:R171-R174, 1998; Schummer, Biotechniques 23:1087-1092, 1997; Kern, Biotechniques 23:120-124, 1997; Solinas-Toldo, Genes, Chromosomes & Cancer 20:399-407, 1997; Bowtell, Nature Genetics Supp. 21:25-32, 1999. See also published U.S. patent applications Ser. Nos. 20010018642; 20010019827; 20010016322; 20010014449; 20010014448; 20010012537; 20010008765. The CGH methods of the invention can be performed using a variety of commercially available CGH arrays, as well as custom designed arrays, that can be commercially fabricated. Examples of commercially available high density arrays and kits include those available from Agilent Technologies for human, mouse, and rat genomic analysis (e.g., G441 IB and G4412A), custom tiling arrays manufactured by Nimblegen (Roche) and whole-genome tiling arrays made by Affymetrix.

In one embodiment, the present invention provides a novel high density array for the detection of a balanced translocation/rearrangement associated with leukemia. In certain embodiments, the high density arrays of the present invention are useful for the diagnosis, for providing a prognosis, or for genotyping a leukemia, such as a myeloid leukemia or a lymphoma. In a particular embodiment, the invention provides an array for detecting the loci found in Table 1. In a particular embodiment, the present invention provides an AML high density array as outlined in Table 1.

The resolution of array-based CGH is primarily dependent upon the number, size and map positions of the nucleic acid elements within the array, which are capable of spanning the entire genome. In a particularly advantageous embodiment of the present invention, oligonucleotide nucleic acid elements are used to form microarrays at tiling density. See, e.g., Mockler, T. C. and J. R. Ecker, Genomics 85: 1 (2005); Bertone, P., M. Gerstein, et al, Chromosome Research, 13: 259 (2005).

For example, oligonucleotide probes can be densely spotted on to a small chip surface tiled in an 3, 4, 5, 6, 7, 8, 9, 10, 11, or 12 basepair(bp) spacing resolution and the oligonucletide probes cover the promoter, introns and exons of each targeted gene and in addition at least approximately 5, 10, 15, 20, 25, 50, 100, 200, 300, 400, 500, 600, 700, 800, 900, 1000 or any number in between basepairs upstream and downstream to the targetted genes are included in the design. The approach supplements the standard microarray analysis platform as the standard platform cannot detect balanced rearrangements. The tiling array also detects the signal ratio for copy number changes i.e., deletions or duplications in the targetted regions. Through the use of high density tiled array with spacing between 3 nd 12 base pairs, false positive can be eliminated by discarding single probe drop offs and identifying drop offs occur at multiple probes. Additionally, by limiting probe length to between 25 and 80 base pairs reduces binding of probes over a break point despite poor hyribdrization at multiple nucleotides.

a) Primers and Probes

“Primers” are a subset of probes which are capable of supporting some type of enzymatic manipulation and which can hybridize with a target nucleic acid such that the enzymatic manipulation such as the priming of the synthesis of a polynucleotide n an amplification reaction can occur. A primer can be made from any combination of nucleotides or nucleotide derivatives or analogs available in the art which do not interfere with the enzymatic manipulation. Typically a primer comprises fewer than about 100 nucleotides and preferably comprises fewer than about 30 nucleotides. Exemplary primers range from about 5 to about 25 nucleotides.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

The phrase “selectively (or specifically) hybridizes to” refers to the binding, duplexing, or hybridizing of a molecule only to a particular nucleotide sequence under stringent hybridization conditions when that sequence is present in a complex mixture.

Parameters for selective hybridization between two nucleic acid molecules are well known to those of skill in the art. For example, in some embodiments selective hybridization conditions can be defined as stringent hybridization conditions. The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993).

Thus, it is understood that stringency of hybridization can be controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Those of ordinary skill will readily recognize that alternative hybridization and wash conditions can be utilized to provide conditions of similar stringency.

For example, for PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

Any of a number of previously described methods for carrying out comparative genomic hybridization may be used in the practice of the present invention, such as those described in U.S. Pat. Nos. 6,197,501; 6,159,685; 5,976,790; 5,965,362; 5,856,097; 5,830,645; 5,721,098; 5,665,549; 5,635,351; Diago, Am. J. Pathol. 158:1623-1631, 2001; Theillet, Bull. Cancer 88:261-268, 2001; Werner, Pharmacogenomics 2:25-36, 2001; Jain, Pharmacogenomics 1:289-307, 2000.

In some cases, prior to the hybridization of a specific probe of interest, it is desirable to block repetitive sequences. A number of methods for removing and/or blocking hybridization to repetitive sequences are known {see, e.g., WO 93/18186). As an example, it may be desirable to block hybridization to highly repeated sequences such as AIU sequences. One method to accomplish this exploits the fact that hybridization rate of complementary sequences increases as their concentration increases. Thus, repetitive sequences, which are generally present at high concentration will become double stranded more rapidly than others following denaturation and incubation under hybridization conditions. The double stranded nucleic acids are then removed and the remainder used in hybridizations. Methods of separating single from double stranded sequences include using hydroxyapatite or immobilized complementary nucleic acids attached to a solid support, and the like. Alternatively, the partially hybridized mixture can be used and the double stranded sequences will be unable to hybridize to the target.

The identification of translocation partners of known genetic loci and the determination of translocation breakpoints is based on a determination of the pattern and intensity of hybridization of labeled probes to one or more nucleic acid elements of the microarray. Typically, the position of a hybridization signal on an array, the hybridization signal intensity, and the ratio of intensities, produced by detectable labels associated with a sample or test probe and a reference probe is determined. The determination of an element that hybridizes to the sample or test probe, but not to the reference probe, identifies the sequence contained within that element as a translocation partner of the known genetic locus. Identical hybridization patterns between the test probe and the reference probe indicate that the tested sample does not contain a translocation at the known genetic locus.

When tiling density microarrays are used, the translocation breakpoints can be determined by ascertaining where in a series of microarray elements representing contiguous genomic segments, hybridization commences or ends (i.e., the drop off). In the case of a balanced translocation/rearrangement, hybridization will begin at a particular DNA sequence within a gene distinct from the known genomic locus. The sequence embodied by the first element in a contiguous sequence of the distinct gene identifies that sequence as representing the breakpoint within the second gene. Conversely, with respect to the known genomic locus, the element within a contiguous sequence where hybridization ends marks that element as representing the translocation breakpoint within the known genomic locus. Thus, where a balanced reciprocal translocation/rearrangement occurs, there will be two drop offs detected, one for each loci involved in the translocation.

Moreover, typically, the greater the ratio of the signal intensities on a target nucleic acid segment, the greater the copy number ratio of sequences in the two samples that bind to that element. Thus comparison of the signal intensity ratios among target nucleic acid segments permits comparison of copy number ratios of different sequences in the genomic nucleic acids of the two samples.

4. Labels and Fluorochromes

As disclosed herein, probes employed by the disclosed methods may be labeled during the course of amplification or after amplification has occurred. It is further understood, that multiple detectable labels may be used to distinguish one sample from another. Thus, for example, the methods disclosed herein can comprise a first and second label wherein either the first or second label is used to identify a test sample and the remaining label is used to identify a reference sample.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO- TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis- BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO- PRO 3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-genrating molecule or moiety, allowing detection of the tertiary immune complexes thus formed. This system can provide for signal amplification.

Labels can be incorporated in a separate step after amplification such as linear amplification by oligonucleotide (random hexamers) mediated primer extension with a DNA polymerase. With this protocol, both the original genomic DNA samples and the linear amplification products will give rise to labeled probes that generate signals. After hybridization, the resulting data will yield information on both chromosomal aberrations from differential genomic DNA signals as seen with normal aCGH, but also reveal chromosomal rearrangements coming from differential signals arising from the linear amplification products. If labels are incorporated simply in the linear amplification products, as would happen if the labeled dNTPs were included in the linear amplification step, then only transloactions would be revealed and not chromosomal abnormalities like amplifications and deletions.

5. Nucleic Acids

The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modifcation, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modiifcations also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones;formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the nucleic acid molecules disclosed herein. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

6. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

7. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, disclosed herein are computer readable mediums comprising the sequences and information regarding the sequences set forth herein.

8. Kits

The present invention also provides kits to facilitate and/or standardize the CGH methods provided herein. Materials and reagents for executing the various methods of the invention can be provided in kits to facilitate these methods. As used herein, the term “kit” refers to a combination of articles that facilitate a process, assay, analysis, diagnosis, prognosis, or manipulation. Thus, in one aspect, disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. Accordingly disclosed herein are kits for use in the detection of a balanced chromosomal translocation/rearrangement, the kit comprising: (a) an array for the detection of product and (b) the reagents needed for dye labeling and CGH microarray hybridization.

In one embodiment, the kits provided by the present invention may comprise a nucleic acid primer for whole genome amplification to increase the amount of sample prior to hybridization or the linear amplification of a genomic locus implicated in balanced translocation/rearrangement. Thus, also disclosed are kits further comprising one or more primers for the amplification of the reference and test samples prior to hybridization or amplification of a genomic locus implicated in a translocation In certain embodiments, the kits may comprise a primer mix for the amplification of multiple genomic loci. In other embodiments, the kits of the invention may comprise a high density tiling array for use in CGH analysis of balanced chromosomal translocations/rearrangements. In certain embodiments, the present invention provides kits useful for the diagnosis, or prognosis of a disease characterized by a balanced translocation/rearrangements, in particular embodiments, the disease is a cancer, such as a lymphoma or a leukemia. In another embodiment, the present invention provides a kit comprising a high density tiling array for the detection of a balanced translocation/rearrangements associated with a myeloid leukemia.

The compositions disclosed herein and the compositions necessary to perform the disclosed methods can be made using any method known to those of skill in the art for that particular reagent or compound unless otherwise specifically noted.

C. Methods of Using the Compositions

1. Methods of Diagnosis

The disclosed compositions can also be used diagnostic tools related to diseases where uncontrolled cellular proliferation occurs such as cancers and precancerous conditions. The diagnosis is made through the detection of balanced reciprocal translocations/rearrangements occurring at know fusion sites such as with BCR and ABL1. For example, a diagnosis of leukemia can be made by performing the disclosed methods on an array designed for the detection of translocations in genes associated with Acute Myeloid Luekemia (AML), Acute lymphoblastic leukemia, and/or Chronic myelogenous leukemia (CML). A drop off is detected at sites of a known fusion partners showing the presence of a balanced reciprocal translocation/rearrangement which resulted in the fusion. Thus, disclosed herein are method of diagnosing of a disease or condition associated with chromosomal rearrangement in a test sample comprising (a) isolating a first genomic DNA from cells of a test sample and a second genomic DNA from cells of a reference sample; b) hybridizing the test and reference DNA products to a tiling density DNA microarray comprising genomic DNA sequences, wherein the there is a 3-12 base pair overlap of DNA oligomeric probes; and (c) comparing the pattern and extent of hybridization of the test amplified DNA product with the reference amplified DNA product to the DNA microarray; wherein a patterned loss and recovery of hybridization of the amplified test sample DNA product over the amplified reference sample DNA product to a DNA microarray element indicates the presence of chromosomal rearrangement; and wherein the presence of a chromosomal rearrangement at a location associated with a disease or condition indicates the presence of the disease or condition. Also disclosed herein are methods wherein whole genome amplification is applied to the test and reference DNA samples prior amplification. It is further disclosed that rather than whole genome amplification, linear amplification can be used prior to the hybridization step.

A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, Ewing sarcoma and other type of sarcomas, hypoxic tumours, myelomas, AIDS-related lymphomas or sarcomas, metastatic cancers, or cancers in general.

A representative but non-limiting list of cancers and precancerous conditions that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, large bowel cancer, hematopoietic cancers; testicular cancer; colon and rectal cancers, prostatic cancer, cervical and anal dysplasias, other dysplasias, severe dysplasias, hyperplasias, atypical hyperplasias, neoplasias, and pancreatic cancer.

Examples of translocations that are known to be involved with various diseases include, without limitation, t(2;5)(p23;q35)—anaplastic large cell lymphoma; t(8;14)—Burkitt's lymphoma (c-myc); 49;22)(q34;q1 1)—Philadelphia chromosome, CML, ALL; t(11;14)—Mantle cell lymphoma (Bcl-I); t(11;22)(q24;q1 1.2-12)—Ewing's sarcoma; t(14;18)(q32;q21)—follicular lymphoma (Bcl-2); t(17;22)—dermatofibrosarcoma protuberans; t(15; 17)—acute promyelocytic leukemia; t(1; 12)(q21;p 13)—acute myelogenous leukemia; t(9;12)(p24;p13)—CML, ALL (TEL-JAK2); t(X;18)(p11.2;q11.2)—Synovial sarcoma; t(1;11)(q42.1;q14.3)—Schizophrenia; t(12;15)(p13;q25)—(TEL-TrkC); acute myeloid leukemia, congenital fibrosarcoma, secretory breast carcinoma.

Accordingly, the present invention also provides methods of predicting, diagnosing, or providing prognoses of diseases that are caused by chromosomal rearrangements, particularly chromosomal translocations, by detecting the presence of a chromosomal translocation and determining the identity of the translocation partners. For example, if a diagnosis of AML is desired, primers and probes for hybridization to a human microarray testing for balanced reciprocal translocations/rearrangements between BCR and ABL1 are used. Using the methods of the invention, a diagnosis of AML would be indicated if hybridization “drop-off” was observed between the test and reference samples.

2. Methods of Using the Compositions as Research Tools

The disclosed compositions can be used as discussed herein as either reagents in micro arrays or as reagents to probe or analyze existing microarrays. The disclosed compositions can be used in any known method for isolating or identifying single nucleotide polymorphisms or balanced reciprocal translocations/rearrangements. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Microarray for Detection of Balance Reciprocal Translocation in Oncology

The specific balanced reciprocal translocation first identified in human cancers was t(9;22)(q34;q11), resulting in the formation of Philadelphia chromosome (derivative chromosome 22). The molecular characterization of t(9;22) in chronic myelogeneous leukemia (CML) revealed a fusion of the BCR gene on chromosome 22 and ABL1 gene on chromosome 9 with no gain or loss of genetic material. This was the basis for understanding the pathogenetic significance of translocations and gene fusions in the origin of cancer. This discovery lead to better understanding of the mechanisms underlying translocations and fusion genes in cancer and cytogenetic analysis became a valuable tool to map genes associated with oncogenesis. Presently, cytogenetics or chromosome analysis has a vital role in cancer diagnosis and prognosis. As a result, the information on chromosome aberrations in cancers has steadily increased over the past decades. Such cytogenetic characterization has been of the greatest importance in detecting genes associated with tumorigenesis and has, to date, led to the identification of approximately 300 genes involved in fusions in both benign and malignant neoplastic disorders. These genes represent a substantial proportion of all mutated genes that have been implicated in oncogenesis.

Comparative genomic hybridization (CGH) is perhaps the most significant technical development in the molecular cytogenetics period, and has contributed considerably to the further understanding of the cancer genome. Over recent years, the chromosome study has been largely superseded by array CGH (aCGH) in which changes in copy number can be mapped to the DNA sequence at a high resolution. However; one of the major problems encountered with translocations or fusion genes detection using aCGH is that it cannot detect balanced rearrangements as is the case with fusion genes; with no gain or loss at the DNA sequence level. Prior to the present disclosure, all the microarray platforms that are available for cancer cytogenetics markers can only target copy number changes i.e., losses or gains of genetic material and do not detect presence of fusion gene sequences as these are balanced. So far, over 300 fusion genes have been identified in cancers and the first designer drug and most effective therapy for CML i.e., Imatinib (commonly known as Gleevac) was based on the knowledge of fusion of the BCR and ABL1 genes. Given recent improvements in quality and expense of custom designed microarrays, the goal to implement a sensitive and cost-effective assay to detect balanced reciprocal translocations/rearrangements is achievable.

For example, the microarray can be a custom designed high density tiling microarray chip (BCRONC/RS) on Roche/Nimblegen platform. Micro array chips can include designing probes for 50 genes that are known to be involved in balanced translocations/rearrangements associated with malignant states. At least 10 cases of CML, acute myeloid leukemia (AML) and Acute lymphoblastic leukemia (ALL) with known translocation between BCR and ABL1 resulting in the fusion genes with variable breakpoint regions can be run on the Nimblegen/Roche customized panel (BCRONC/RS) for BCR/ABL1 genes. Identifying translocations of the BCR/ABL1 gene is important for diagnosis and to provide prognostic information. However, cytogenetic analysis does not always detect the translocations either due to poor morphology of the chromosomes or failure of the abnormal clone to grow in culture and the cryptic or submicroscopic nature of the rearrangement at rare occasions make detection difficult. The disclosed array is designed so balanced reciprocal translocations/rearrangements are detected regardless of the breakpoint or partner gene as some genes have multiple partners. In addition, copy number changes i.e., gains or losses in the targeted regions are detected. In hematological malignancies, additional changes are suggestive of either clonal evolution or progression. This assay detects additional changes that are cryptic or submicroscopic in nature and not detected by conventional cytogenetics either due to poor chromosome morphology or size of the chromosomal abnormalities.

The disclosed methods are applicable to all current available platforms such as Roche/Nimblegen CGH array, AgilentCGH array, Affymetirx SNP array and Illumina SNP array. The content of the BSONCO/RS chip has been determined. The sequences of 50 genes have been added to the BSONCO/RS chip design file. The Roche/Nimblegen instruments are in house and with trained knowledgeable research scientists and specialists, the validation can be completed in two to three months.

a) Methods

### (1) BCRONCO/RS Array Design and Principle

A 50 genes panel has selected to target by this microarray. The genes were selected based on the current knowledge of cancer cytogenetics (WHO). For all 50 genes, NM reference, chromosome number, strand orientation, coordinates 500 bp upstream and downstream of the gene based on REFseq information available through the UCSC genome browse and UCSC tracking information provided. All the coordinates are based on build hg18. The longer transcript that encodes the longer isoform for each gene was selected. For details of genes included in the design please see the Table 1.

The reference sequence file obtained from UCSC Genome Browser. A tiling strategy has been taken for the aCGH probe design in an average of 5 bp spacing. The 60 mers oligonucleotide probes have been designed and selected using the oligonucleotide selection programs. The probes have been designed with isothermal condition in Tm at 65° C. for equal hybridization efficiency. Then the probes have been searched for specificity using BLAST via the NCBI standalone blast program. Furthermore, the repeat elements such as SINE and LINE and low copy number (LCN) repeats region have been avoided to place the probes. The array design and probe locations can be viewed in the UCSC Genome Brower.

In principal, the array probes hybridize to target genomic DNA when the probe sequence is perfect match to the complementary targets. Because of the copy number nature for balanced reciprocal translocations/rearrangements, the BCRONCO/RS chip detects two copies of both genes which are involved in genome rearrangement. When the tiled probes reach the translocation breakpoint, due to the mismatch of the probe to the targeted DNA sequences, the probes do not hybridize to the targets and the results show the probe drop off; the same as the deletion occurred in the region. After the probes tiled through the translocation breakpoint, the probe detects diploidy again. Therefore, the microarray results show the “U or crescent” shape in both translocation/rearrangement partner genes. Because the “U or crescent” shape pattern was only seen in the probes across the translocation breakpoint, the size of the “U or crescent” shape pattern is expected much smaller than the actual deletions.

### (2) Method of BCRONCO/RS Array

DNA was isolated from the bone marrow aspirate using ArchivePure DNA Blood Kit (5 Prime, Inc., Gaithersburg, Md.) per the manufacturer's instructions. Microarray analysis was performed using the Custom designed Roche/Nimblegen 3X720K chip (Roche/Nimblegen) following the manufacturer's protocol.

### (3) NimbleGen CGH Sample Preparation and Labeling Protocol

Targeted samples (1.0 ug of DNA) and reference gDNA are randomly fragmented by heat at 95° C. for 10 mins. Following the fragmentation step, the DNA samples are labeled with Cyanine3 (Cy3) on the test sample DNA and Cyanine5 (Cy5) with reference DNA. Labeling is achieved through the hybridization and extension of Cy-dye-labeled random nonamers (provided by Roche NimbleGen Inc, Madison, Wis.). Exo-Klenow fragment is used to extend the labeled nonamers with unlabeled nucleotides (kit provided by Roche NimbleGen Inc, Madison, Wis.). The labeling reaction is carried out for 2 hrs at 37° C. and stopped with the addition of 0.5M EDTA. The reactions are pelleted by centrifugation and rinsed with 80% ethanol and resuspended in water. The concentration of the purified samples is determined using a NanoDrop ND-1000 spectrophotometer.

### (4) Hybridization and Wash Protocol

The Cyanine-labeled test sample (Cy3) and reference sample (Cy5) are combined (31 ug each) and dried down by vacuum centrifugation. The samples are rehydrated in 5.6 ul of NimbleGen Hybridization Buffer. They are denatured at 95° C. for 5 min, and then cooled at 42° C. 18 ul of this mixture is loaded into a NimbleGen 3X720 K custom array and hybridizations are carried out for 48 hr at 42° C. with active mixing in the Roche NimbleGen 4-Bay Hybridization System. The arrays are washed using a Roche NimbleGen Wash Buffers (Roche NimbleGen Inc, Madison, Wis.) and immediately dried down by centrifugation.

### (5) Signal Scanning and Feature Extraction

Microarray slides are scanned at 5-μm resolution using Agilent Technologies G2505B Microarray Scanner (Agilent Technologies, Santa Clara, Calif.) with Roche NimbleGen array compatible software. The scanner performs simultaneous detection of Cyanine-3 and Cyanine-5 signal on the hybridized slide. Data captured from the scanned microarray image are saved as a TIF files. Data are extracted from scanned images using NimbleScan 2.5 extraction software, which allows for automated grid alignment, extraction, and generation of data files.

### (6) BCRONCORS Array Data Analysis

Data analysis was performed with SignalMap (Roche Nimblegene). Deletion/amplification and gene rearrangement breakpoints are determined by automated segmentation analysis of data sets as follows: Analysis within the NimbleScan 2.5 program is done with the segMNT algorithm. This process normalizes the signal intensities of the test vs. reference. For the 3X720K custom array, the log 2 ratios are averaged at window sizes corresponding to 5× and 10× the median probe spacing. Log ratios at each position are averaged before window averaging. Both the unaveraged and window-averaged log 2 ratios are used to produce the final segmentations. The deletion and duplication detection are based on the sequences of 50 genes have been added to the BSONCO/RS chip using May 2004 assembly of the UCSC human genome hg 18 database (http://genome.ucsc.edu) and the deletion/duplication and gene rearrangements are checked in the Database of Genomic Variants (http://projects.tcag.ca/variation/) for the known CNVs. The log 2 Ratio <−0.4 with the minimum of 8 consecutive probes is considered a deletion or gene rearrangement detected and log 2 ratio >0.4 with a minimum of 8 consecutive probes is considered a duplication.

b) Results

20 samples previously detected gene rearrangement by either karyotyping or FISH have been tested with ONCO array. All the translocation can be detected as the “U or crescent” shape from probe drop-offs. Below are two representative examples:

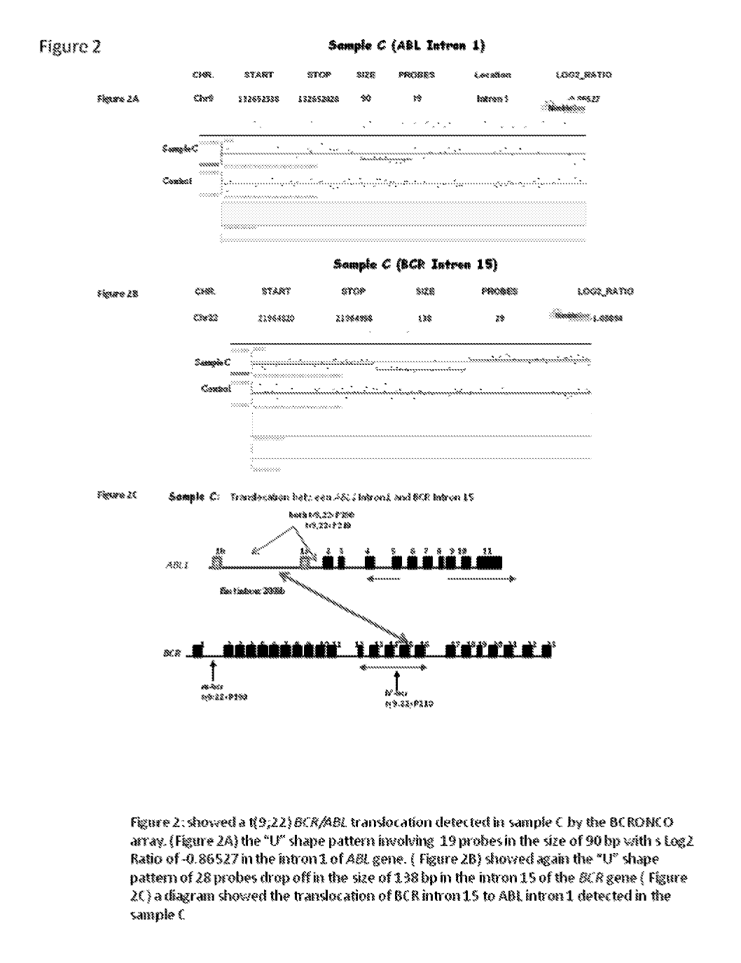

Sample C is a patient diagnosed with CML and FISH analysis showed a gene rearrangement between BCR on chromosome 22 and ABL1 on chromosome 9 resulting in a Philadelphia Chromosome. The BCRONCO/RS arrays showed the “U or crescent” shape pattern of probe “drop-off” in the chromosomal region start at nucleotide position 21964820 and end at 21964958 on Chromosome 22. There are 29 probes drop-off; size of the region of deletion was approximately 138 bp and the Log 2 Ratio was −1.058. It showed that the “U or crescent” shape pattern of probe drop-off were in the intron 15 of the BCR. On the other hand, the probe “drop-off” was also seen in chromosome 9 at the region of staring at nucleotide position 20337285 and ending at position 20337341. It was included 15 probes with the size of 56bp region for probe e drop-off and the Log 2 Ratio was −1.6654. The probes were located in the intron 1 of the ABL1 gene (see FIG. 2).

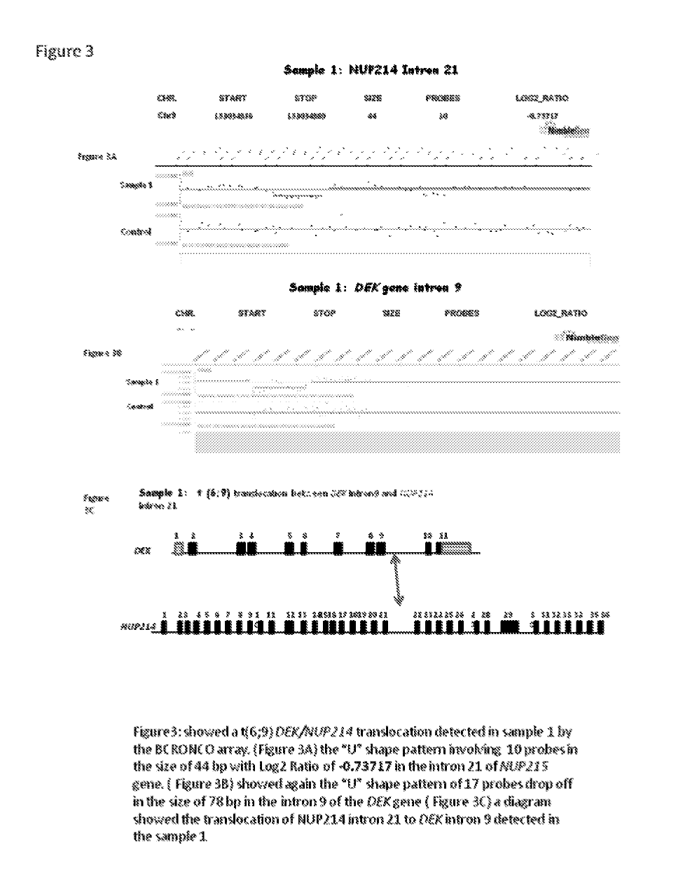

Sample 1 is the patient with AML and FISH analysis detected a balance translocation between the chromosome 6p23 and chromosome 9q34 involving the DEK in chromosome 6 and NUP214(CAN) in chromosome 9. The BCRONCO/RS array showed the “U or crescent” shape pattern of probe drop-offs on chromosome 6 in the region start at nucleotide position of 18369236 and end at 18369314. There were 17 probes drop-off in the intron 9 of the DEK and the Log 2 ratio was −0.706. On the translocation partner chromosome 9, the “U or crescent” shape pattern of probe drop-off was detected on the chromosomal region starting at nucleotide position of 133034836 and ending at position 133034880 in the intron 21 of NUP214 gene. It showed 10 probe drops off for the size of 44 bp and the Log 2 Ratio was −0.737 (see FIG. 3).

TABLE 1
The 50 genes in the BCRONCO array

| Genes | Reference | Chromosome | Start | Stop | Size |
|---|---|---|---|---|---|
| TAL1 | NM_003189 | 1 | 47,454,550 | 47,468,030 | 13,480 |
| RBM15(OTT) | NM_022768 | 1 | 110,683,468 | 110,690,826 | 7,358 |
| TPM3/NEM1 | NM_153649 | 1 | 152,394,404 | 152,422,349 | 27945 |
| PBX1 | NM_002585 | 1 | 162,795,426 | 163,087,669 | 292,243 |
| ALK | NM_004304 | 2 | 29,269,144 | 29,997,936 | 728,792 |
| IGK | uc010fhk.1 | 2 | 88,978,123 | 88,978,415 | 292 |
| RPN1 | NM_002950 | 3 | 129,821,503 | 129,852,409 | 30,906 |
| MECOM (EVI1) | NM_001164000 | 3 | 170,283,981 | 170,348,216 | 64,235 |
| BCL6 | NM_001130845 | 3 | 188,921,859 | 188,936,979 | 15,120 |
| FGFR3 | NM_001163213 | 4 | 1,764,837 | 1,780,397 | 15,560 |
| FIP1L1 | NM_030917 | 4 | 53,938,577 | 54,020,859 | 82,282 |
| CHIC2 | NM_012110 | 4 | 54,570,715 | 54,625,545 | 54,830 |
| PDGFRA | NM_006206 | 4 | 54,790,021 | 54,859,169 | 69,148 |
| AFF1 | NM_005935 | 4 | 88,147,177 | 88,281,215 | 134,038 |
| (AF4)/MLLT2 | | | | | |
| IL3 | NM_000588 | 5 | 131,424,246 | 131,426,795 | 2,549 |
| PDGFRB | NM_002609 | 5 | 149,473,595 | 149,515,615 | 42,020 |
| DEK | NM_001134709 | 6 | 18,332,381 | 18,372,778 | 40,397 |
| C-MYB | NM_001161660.1 | 6 | 135,544,146 | 135,582,004 | 37,858 |
| MLLT4 (AF6) | NM_005936 | 6 | 167,970,520 | 168,095,791 | 125,271 |
| FGFR1 | NM_023108 | 8 | 38,400,009 | 38,445,509 | 45,500 |
| ETO1(RUNX1T1) | NM_175636 | 8 | 93,040,328 | 93,099,084 | 58,756 |
| C-MYC | NM_002467 | 8 | 128,817,497 | 128,822,860 | 5,363 |
| MLLT3/AF9 | NM_004529 | 9 | 20,334,968 | 20,612,514 | 277,546 |
| CDKN2A | NM_058197.4 | 9 | 21,957,776 | 21,996,246 | 38,470 |
| ABL1 | NM_007313 | 9 | 132,579,089 | 132,752,883 | 173,794 |
| NUP214 (CAN) | NM_005085 | 9 | 132,990,802 | 133,098,912 | 108,110 |
| TET1 | NM_030625 | 10 | 69,990,123 | 70,124,245 | 134,122 |
| CCND1 | NM_053056 | 11 | 69,165,054 | 69,178,423 | 13,369 |
| ATM | NM_000051 | 11 | 107680612 | 107691848 | 11,236 |
| MLL | NM_005933 | 11 | 117,812,415 | 117901146 | 88,731 |
| ETV6 (tel) | NM_001987 | 12 | 11,694,055 | 11,939,592 | 245,537 |
| FOXO1 | NM_002015 | 13 | 40,027,801 | 40,138,734 | 110,933 |
| IGH@ | uc001ysw.1 | 14 | 105180450 | 105997769 | 817,319 |
| PML | NM_033238 | 15 | 72,074,067 | 72,127,208 | 53,141 |
| MYH11 | NM_002474 | 16 | 15,704,493 | 15,858,388 | 153,895 |
| CBFB | NM_001755 | 16 | 65,620,551 | 65,692,459 | 71,908 |
| MAP (c-maf) | NM_005360 | 16 | 78,185,247 | 78,192,123 | 6,876 |
| TP53 | NM_000546 | 17 | 7,512,445 | 7,531,588 | 19,143 |
| RARA | NM_001024809 | 17 | 35,751,868 | 35,767,420 | 15,552 |
| MALTI | NM_173844 | 18 | 54,489,598 | 54,568,350 | 78,752 |
| BCL2 | NM_000633 | 18 | 58,941,559 | 59,137,593 | 196,034 |
| TCF3(E2A) | NM_003200 | 19 | 1,560,293 | 1,603,326 | 43,033 |
| MLLT1(ENL) | NM_005934 | 19 | 6,161,392 | 6,230,959 | 69,567 |
| ELL | NM_006532 | 19 | 18,414,474 | 18,493,937 | 79,463 |
| BCL3 | NM_005178 | 19 | 49,943,818 | 49,955,141 | 11,323 |
| MAFB (KRML) | NM_005461 | 20 | 38,747,931 | 38,751,290 | 3,359 |
| RUNX1 | NM_001754 | 21 | 35,081,968 | 35,343,465 | 261,497 |
| BCR | NM_004327 | 22 | 21,852,552 | 21,990,224 | 137,672 |
| IGL@/IGLL1 | NM_152855 | 22 | 22,245,313 | 22,252,495 | 7,182 |
| SRY | NM_003140 | Y | 2,714,896 | 2,715,792 | 896 |
| | | | | | 5,101,565 |
| | | | | | 7.085506944 |

Additional 500 bp of upstream and downstream of the gene has been included.

What is claimed is:

1. A method of detecting the presence of a chromosomal rearrangement in a test sample comprising:
(a) isolating a first genomic DNA from cells of a test sample and a second genomic DNA from cells of a reference sample;
(b) hybridizing the test and reference DNA products to a tiling density DNA microarray comprising genomic DNA sequences, wherein the there is a 3-12 base pair overlap of DNA oligomeric probes; and
(c) comparing the pattern and extent of hybridization of the test DNA product with the reference DNA product to the DNA microarray;
wherein a patterned loss and recovery of hybridization of the test sample DNA product over the reference sample DNA product to a DNA microarray element indicates the presence of chromosomal rearrangement.

2. The method of claim 1, wherein the patterned loss and recovery of hybridization occurs at the translocation breakpoint.

3. The method of claim 1, wherein the rearrangement is a balanced reciprocal translocation.

4. The method of claim 1, wherein the first and second genomic DNA are labeled with a first and second detectable label, respectively.

5. The method of claim 4, wherein the detectable labels are incorporated using random priming, nick translation or end labeling.

6. The method of claim 5, wherein the first and second detectable label are fluorescent labels.

7. The method of claim 1, wherein the cell of the test sample is a tumor cell and the reference cell is a normal cell.

8. The method of claim 7, wherein the tumor cell is a lymphoma or leukemia or solid tumors.

9. The method of claim 1, wherein the oligomeric probes are 50-80 base pairs in length.

10. The method of claim 1, wherein the oligomeric probes on the microarray cover the sequence of the target gene comprising at least 100 base pairs upstream and downstream of the open reading frame.

11. The method of claim 1, wherein the spacing between contiguous probes on a array is between 3 and 12 base pairs.

12. The method of claim 1, wherein the method further comprises the detection of a second chromosomal rearrangement; wherein the first and second rearrangement identify fusion partners associated with a balanced reciprocal translocation/rearrangement.

13. A method of diagnosing of a disease or condition associated with chromosomal rearrangement in a test sample comprising:
(a) isolating a first genomic DNA from cells of a test sample and a second genomic DNA from cells of a reference sample;
(b) hybridizing the test and reference DNA products to a tiling density DNA microarray comprising genomic DNA sequences, wherein the there is a 3-12 base pair overlap of DNA oligomeric probes; and
(c) comparing the pattern and extent of hybridization of the test DNA product with the reference DNA product to the DNA microarray;
wherein a patterned loss and recovery of hybridization of the test sample DNA product over the reference sample DNA product to a DNA microarray element indicates the presence of chromosomal rearrangement; and wherein the presence of a chromosomal rearrangement at a location associated with a disease or condition indicates the presence of the disease or condition.

14. The method of claim 13, wherein the patterned loss and recovery of hybridization occurs at the translocation breakpoint.

15. The method of claim 13, wherein the rearrangement is a balanced reciprocal translocation/rearrangements.

16. The method of claim 13, wherein the method further comprises the detection of a second chromosomal rearrangement; wherein the first and second rearrangement identify fusion partners associated with a balanced reciprocal translocation/rearrangement.

17. The method of claim 13, wherein the disease or condition is selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, large bowel cancer, hematopoietic cancers; testicular cancer; colon and rectal cancers, prostatic cancer, cervical and anal dysplasias, other dysplasias, severe dysplasias, hyperplasias, atypical hyperplasias, neoplasias, and pancreatic cancer.

18. The method of claim 1 or 13, wherein the detection is by PCR, sequencing, mass spectrometry, hybridization, or CGH analysis.

19. A kit for use in the detection of a balanced chromosomal translocation/rearrangement, the kit comprising: (a) an array for the detection of product and (b) the reagents needed for dye labeling and CGH microarray hybridization.

20. The kit of claim 19, further comprising one or more primers for amplifying the reference and test samples prior to hybridization.

21. The kit of claim 20, wherein said kit comprises a plurality of primers for the amplification of loci implicated in translocations.

22. An array for CGH analysis of a chromosomal rearrangement associated with a disease.

23. The array of claim 22, wherein said array comprises a plurality of probes specific for at least two loci, wherein the loci are implicated in a chromosomal rearrangement associated with a disease.

24. The array of claim 22, wherein at least one rearrangement associated with a disease is a balanced translocation/rearrangement.

25. The array of claim 22, wherein said disease is lymphoma or leukemia.
